(19) United States
(12) Patent Application Publication
(10) Pub. No.: US 2026/0014178 A1
(43) Pub. Date: Jan. 15, 2026

(54) COMPOSITIONS AND USES THEREOF

(71) Applicant: Optibiotix Limited, York Yorkshire (GB)

(72) Inventors: Stephen O'Hara, York Yorkshire (GB); Sofia Kolida, York Yorkshire (GB)

(21) Appl. No.: 18/992,645

(22) PCT Filed: Jul. 10, 2023
(86) PCT No.: PCT/GB2023/051807

§ 371 Date: Jan. 9, 2025

(30) Foreign Application Priority Data
Jul. 10, 2022 (GB) 2210106.7

(51) Int. Cl. A61K 31/702; A61K 31/733; A61K 33/06; A61K 33/14; A61P 1/14

(52) U.S. Cl. CPC A61K 31/702; A61K 31/733; A61K 33/06; A61K 33/14; A61P 1/14

(57) ABSTRACT

The present invention relates to a composition comprising xylooligosaccharides (XOS), inulin and magnesium (Mg). The composition is particularly suited for use in improving the gastrointestinal microbiota, and maintaining and/or improving gut barrier integrity, and increasing the bioavailability of Mg in an individual. A method of preparing said composition and the use of said composition as a dietary supplement or a medicament are also disclosed.

### FIELD

The present invention relates to compositions and formulations for use in improving the gastrointestinal microbiota composition and activity, and maintaining and improving gut barrier integrity, so as to support the health and wellbeing of an individual. The present invention is particularly useful for older individuals and those individuals involved in amateur and professional sports.

### BACKGROUND

Gastrointestinal microbiota has been shown to confer several health benefit and there is a growing body of evidence linking metabolic diseases with the gut microbiota.

Certain functional fibres are resistant to digestion in the upper gastrointestinal tract, and therefore reach the colon and then undergo at least partial fermentation in the large intestine. If these functional fibres selectively stimulate the growth of beneficial gut microbiota, they are defined as prebiotics. It has been suggested that combining functional fibres may be useful in increasing the diversity of microbiota in the gut. For example, Lecerf, Jean-Michel, et al (2012) British Journal of Nutrition, 108, 1847-1858 describes that XOS in combination with inulin modulated both the intestinal environment and immune status in healthy subjects. EP2467145 discloses the effect of inulin and partially hydrolysed arabinoxylan (AXOS) on inflammation.

To achieve the desired prebiotic effect from inulin, it necessary to consume it in high quantities. However, consuming high quantities of inulin can create physiological issues, such as abdominal pain, and unwanted gas production. It is also difficult to formulate inulin in high doses as it cannot be incorporated into an easily administered capsule or tablet.

It is an object of the present invention to provide a composition which can be used for maintaining and/or improving gastrointestinal microbiota. It is preferred that the composition also helps to maintain and improve gut barrier integrity. It would be beneficial if the composition provided could be in the form of a food supplement, additive or potential treatment in the form of a capsule or tablet. It is a further object of the present invention to provide a composition which can be used to maintain and/or increase the gastrointestinal microbiota of a sports person, and in particular a sports person having a high protein diet. It is a yet further object of the present invention to provide a composition which can be used to maintain and/or increase the gastrointestinal microbiota of an aging person, such as a person who is over 60 years of age. It would also be preferable that the composition could be easily administered as either a medicament or a food supplement or formulated with a food supplement (such as a high protein milkshake or bar).

### SUMMARY OF THE INVENTION

In accordance with an aspect of the present invention, there is provided a composition comprising xylooligosaccharides (XOS), inulin and magnesium (Mg).

The composition will preferably be for use in improving the gastrointestinal microbiota, and maintaining and/or improving gut barrier integrity in an individual.

The improved gastrointestinal microbiota will preferably increase the abundance of desirable microbiota. The microbiota preferably comprises Bifidobacterium and Lactobacillus. More preferably, the microbiota comprises Bifidobacterium.

The improved gastrointestinal microbiota resulting from the composition of the present invention may promote saccharolytic fermentation and/or reduces proteolysis.

The improved gastrointestinal microbiota resulting from the composition of the present invention may also promote improve bowel function.

Advantageously, faecal fermentation experiments conducted by the present inventors show that both XOS and inulin have a significant impact in enhancing faecal Bifidobacterium numbers. The XOS component is rapidly fermented and can enhance Bifidobacterium levels in the proximal-transverse colon, whereas the slowly fermented inulin can support their populations from transverse to the distal colon. The addition of Mg further enhances the health benefits to the individual and the combination of XOS and inulin allow for better bioavailability of Mg. The composition leads to improved bowel function which in turn decreases the presence and concentration of toxic metabolites.

Furthermore, studies have shown that the combination of XOS with inulin has a similar effect as a high dose of inulin. This not only greatly reduces the side effects of consuming inulin in high doses (such as abdominal pain and gas production), but also allows inulin to be incorporated in a wider range of consumable formats (such as capsules or tablets). The present invention therefore provides for a means of reducing the dose of inulin, whilst maintaining similar positive effects of a high dose of inulin.

The individual may be a sports person or an individual undergoing athletic training. The sports person or an individual undergoing athletic training may be undergoing a high protein diet regime.

The term “sports person” or “individual undergoing athletic training” is intended to mean any person who is exerting physical activity with a view to amateur or professional competition, including, but not limited to, those individuals wishing to build muscle mass.

The term “high protein diet regime” is intended to mean an eating regime which incorporates a protein intake exceeding the recommended dietary intake. Such a protein intake will be determined by the sex and mass of the individual. The Dietary Reference Intake (DRI) is generally defined as 0.8 grams of protein per kg of body weight. Generally speaking, the high protein diet regime will comprise the consumption of at least about 1 g of protein per day per kg of body weight. Preferably, the high diet regime will comprise the consumption of at least about 1.4 g of protein per day per kg of body weight. Most preferably, the high diet regime comprises the consumption of about 1.4 g to about 2 g of protein per day per kg of body weight.

Advantageously, the present inventions have found that a composition comprising inulin and XOS increases Bifidobacterium concentration. Bifidobacterium concentrations decline, and Mg levels are lower, in individuals who are undergoing athletic training and who are typically on a high protein diet. The composition of the present invention, can be utilised to maintain Bifidobacterium concentrations, and increase the bioavailability of Mg in those individuals who are undergoing athletic training and who are on a high protein diet. Furthermore, the composition could be incorporated into a protein bar or milkshake so as to help counterbalance the reduction in Bifidobacterium concentrations due to the high protein diet.

Alternatively, the individual may be an aging individual.

The term “aging individual” is intended to mean an individual who is of an age of at least 45 years of age or of at least 60 years of age. Whilst the benefits of consuming the composition will be more appropriate for individuals who are at least 60 years of age, the human body starts to undergo the aging process from 45 years of age onwards so it would be beneficial to consume the composition from 45 years of age. When an individual is at least 60 years of age, they will be subjected to more rapid muscle loss and sarcopenia.

As it is known that there is a significant decline in Bifidobacterium concentrations, and lower Mg levels, in aging individuals, the composition of the present invention, could advantageously be utilised to maintain Bifidobacterium concentrations, and increase the bioavailability of Mg, in those individuals. The composition of the present invention may be utilised to maintain Bifidobacterium concentrations and reduce proteolysis in those aging individuals.

The composition of the present invention, can be utilised to promote saccharolytic fermentation throughout the colon and to reduce proteolysis. Proteolysis produces cellular toxins, carcinogenic and precarcinogenic compounds (ammonia, amines, indoles, phenols and hydrogen sulphide), which further damage the gut barrier. The composition of the present invention can reduce the formation of proteolysis metabolites and enhance their clearance from the body by supporting a regular bowel habit. The promotion of saccharolytic fermentation is applicable to athletes because of high protein intake and insufficient fibre in their diet. The promotion of saccharolytic fermentation is also applicable to aging individuals as studies report that in comparison to young adults, the faecal microbiota of non-institutionalised elderly showed an increase in proteolytic potential, but decreased saccharolytic potential with a low abundance of genes encoding steps in short-chain fatty acids production pathways.

Preferably, the inulin is derived from chicory and/or the XOS is derived from maize. The Mg may be in a number of forms, such as carbonate, chloride, citrate, dictrate, gluconate, glycerophosphate, lactate, oxide, phosphate, dictrate, hydrogen citrate, hydrogen phosphate and sulphate. Preferably, the Mg is in the form of chloride, chloride 6 hydrate, citrate, dictrate or hydrogen citrate.

The inulin and XOS may be present in a ratio in the range of about 4:1 to about 1:1. Preferably, the inulin and XOS may be present in a ratio in the range of about 3:1 to about 1:1. Most preferably, the inulin and XOS are present in a ratio of about 2:1.

The inulin and XOS and Mg may be present in a ratio in the range of a) about 13 to about 3 to about 1; and b) about 4.5 to about 3 to about 1. Preferably, the inulin and XOS and Mg is present in a ratio of about 6.5 to about 3 to about 1.

The inulin may be present in an amount in the range of about 0.6 g to about 4 g. Preferably, the inulin is present in an amount in the range of about 0.6 g to about 1.5 g.

The XOS may be present in an amount in the range of about 0.4 g to about 2.6 g. Preferably, the XOS is present in an amount in the range of about 0.4 g to about 0.5 g.

In certain embodiments, the Mg is present in an amount in the range of about 18 mg to about 120 mg. Preferably, the Mg is present in an amount in the range of about 18 mg to about 85 mg.

In other embodiments, the Mg is present in an amount in the range of about 0.25 g to about 0.75 g. Preferably, the Mg is present in an amount in the range of about 0.4 g to about 0.6 g. Most preferably, the Mg is present in an amount of about 0.5 g. The composition may be formulated so as to provide a daily dose of inulin in an amount in the range of about 2 g to about 12 g. Preferably, the composition is formulated so as to provide a daily dose of inulin in an amount in the range of about 3 g to about 5 g.

The composition may be formulated so as to provide a daily dose of XOS in the range of about 1.4 g to about 8 g. Preferably, the composition is formulated so as to provide a daily dose of XOS in the range of about 1.4 g to about 1.5 g.

In certain embodiments, the composition may be formulated so as to provide a daily dose of Mg in the range of about 55 mg to about 350 mg. Preferably, the composition is formulated so as to provide a daily dose of Mg in the range of about 55 mg to about 250 mg.

In other embodiments, the composition may be formulated so as to provide a daily dose of Mg in the range of about 0.75 g to about 2.25 g. Preferably, the composition may be formulated so as to provide a daily dose of Mg in the range of about 1 g to about 2 g. Most preferably, the composition is formulated so as to provide a daily dose of Mg of about 1.5 g.

The composition may be in the form of a powder, tablet, or capsule.

The composition may further comprise an excipient or carrier compound so as to modify the release profile of the inulin and/or XOS through the intestinal environment.

The composition may be in the form of a food stuff or food additive.

The composition may be for use as a dietary supplement, for example to be blended with foods/drinks or consumed alongside foods/drinks.

The composition may further comprise one or more active ingredients selected from: vitamins, phytochemicals, further minerals, antioxidants, and combinations thereof.

Vitamins may include fat soluble vitamins such as vitamin A, vitamin D, vitamin E, and vitamin and combinations thereof. In some embodiments, vitamins can include water soluble vitamins such as vitamin C (ascorbic acid), the B vitamins (thiamine or B 1, riboflavoin or B25 niacin or B3, pyridoxine or B6, folic acid or B9, cyanocobalimin or B12, pantothenic acid, biotin), and combinations thereof.

Antioxidants may include but are not limited to ascorbic acid, citric acid, rosemary oil, vitamin A, vitamin E, vitamin E phosphate, tocopherols, di-alpha-tocopheryl phosphate, tocotrienols, alpha lipoic acid, dihydrolipoic acid, xanthophylls, beta cryptoxanthin, lycopene, lutein, zeaxanthin, astaxanthin, beta-carotene, carotenes, mixed carotenoids, polyphenols, fiavonoids, and combinations thereof.

Further minerals may include, but are not limited to, sodium, chromium, iodine, iron, manganese, calcium, copper, fluoride, potassium, phosphorous, molybdenum, selenium, zinc, and combinations thereof. If a further mineral is provided, it is preferred that one or more further minerals are selected from either calcium or zinc.

Phytochemicals may include but are not limited to cartotenoids, chlorophyll, chlorophyllin, fiber, flavanoids, anthocyamns, cyaniding, delphinidin, malvidin, pelargonidin, peonidin, petunidin, flavanols, catechin, epicatechin, epigallocatechin, epigailocatechingallate, theaflavins, thearubigins, proanthocyanins, flavonols, quercetin, kaempferol, myricetin, isorhamnetin, flavononeshesperetin, naringenin, eriodictyol, tangeretin, flavones, apigenin, luteolin, lignans, phytoestrogens, resveratrol, isoflavones, daidzein, genistein, glycitein, soy isoflavones, and combinations thereof.

The composition may be for use as a medicament.

In accordance with a further aspect of the present invention, there is provide a method of producing a composition, the method comprising combining xylooligosaccharides (XOS), inulin and Mg.

The composition of the method is preferably for use in improving the gastrointestinal microbiota, and maintaining and/or improving gut barrier integrity in an individual.

The inulin is preferably derived from chicory and/or the XOS is derived from maize.

The inulin and XOS may be combined in a ratio in the range of about 4:1 to about 1:1. Preferably, the inulin and XOS is combined in a ratio in the range of about 3:1 to about 1:1. Most preferably, the inulin and XOS are combined in a ratio of about 2:1.

The inulin and XOS and Mg may be present in a ratio in the range of a) about 13 to about 3 to about 1; and b) about 4.5 to about 3 to about 1. Preferably, the inulin and XOS and Mg is present in a ratio of about 6.5 to about 3 to about 1.

The invention is described below, by way of example only, with reference to the accompanying figures in which:

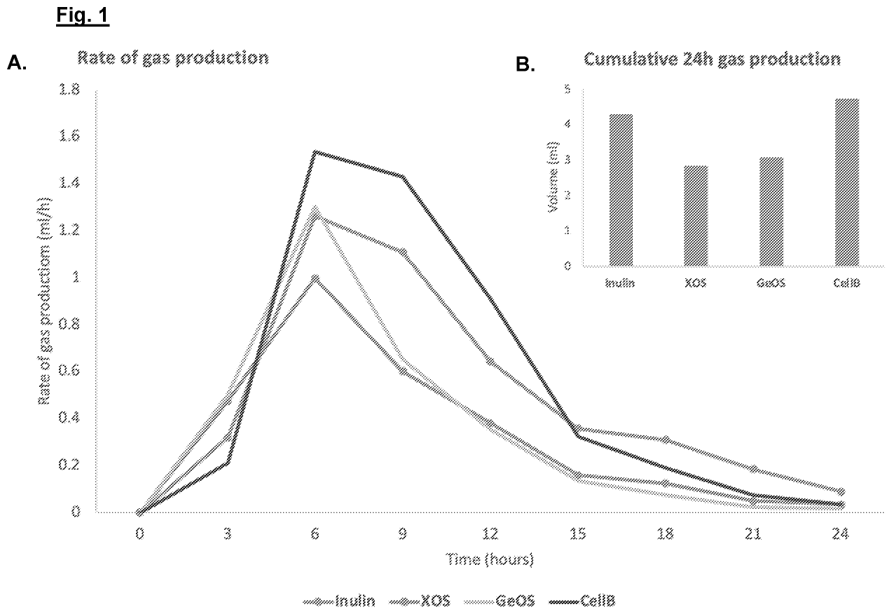

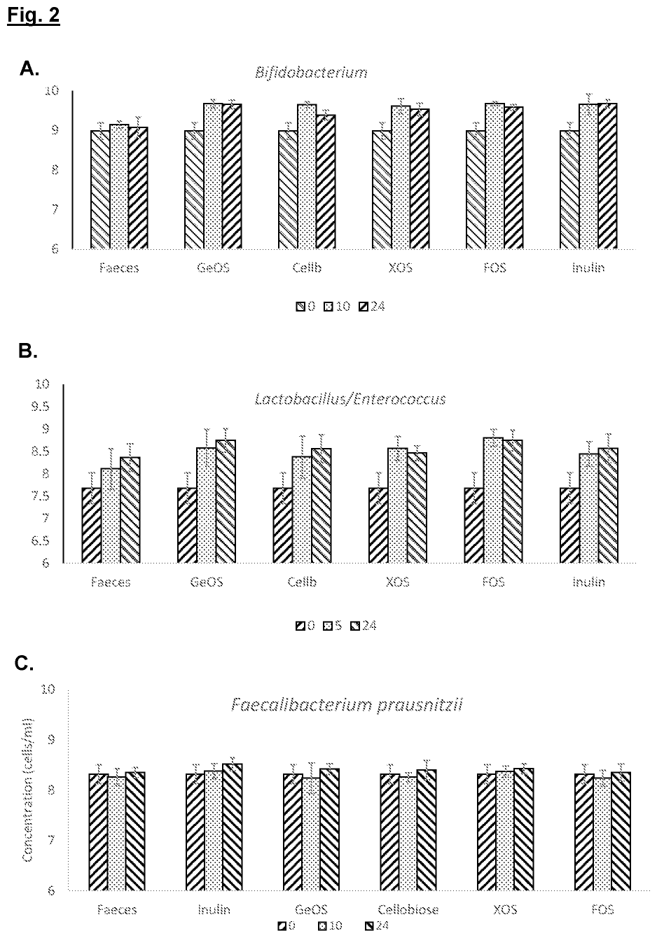

FIG. 1 are graphs showing the impact of XOS and inulin on gas produced by gut microbiome activity. Graph A shows the rate of gas production, whereas graph B shows the cumulative 24 hour gas production;

FIG. 2 are graphs showing the impact of XOS and inulin on the microbial composition of the gut microbiome over the course of 0, 10 and 24 hours. Graph A shows the composition of Bifidobacterium, graph B shows the composition of Lactobacillus/Enterococcus and graph C shows the composition of Faecalibacterium prausnitzii where the Y-axis for all graphs represents the concentration of cells/ml; and

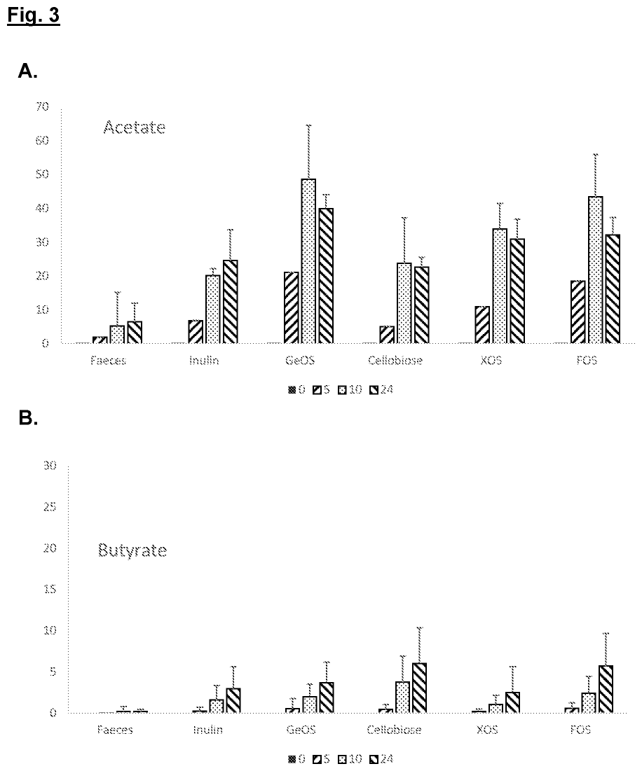

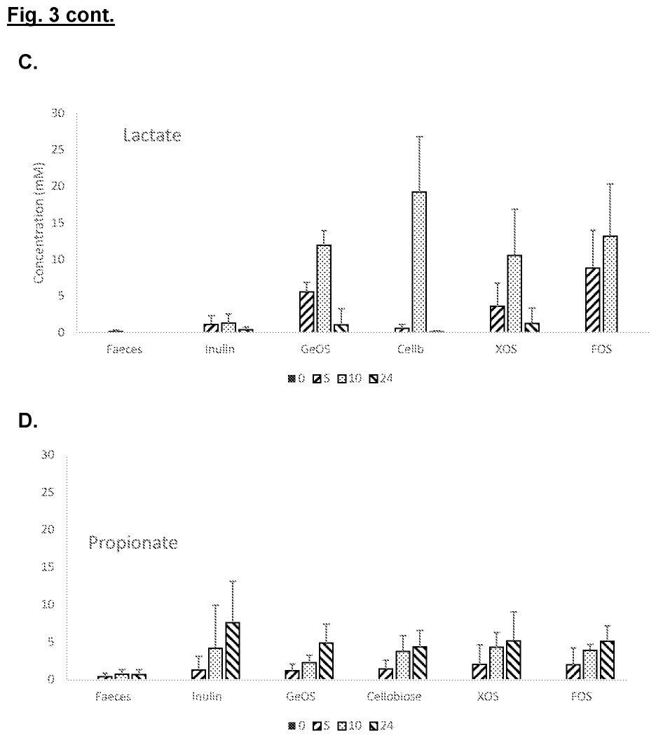

FIG. 3 are graphs showing the impact of XOS and inulin on organic acids produced by the microbial the gut microbiome over the course of 0, 10 and 24 hours. Graph A shows the production of acetate, graph B shows the production of butyrate, graph C shows the production of lactate and graph D shows the production of propionate where the Y-axis for all graphs represents the concentration of organic acids in mM.

### EXAMPLES

### Example 1—In Vitro Evaluation of Dietary Fibres for Inulin and XOS

The impact on faecal microbiome composition and activity of Inulin and XOS was evaluated in vitro.

### Inulin

Inulin is a well established prebiotic with high degree of polymerisation. It can be derived from a number of sources, such as chicory, agave, globe artichoke, Jerusalem artichoke or mango.

Inulin comprises β 1-2 glucose-fructose linkages. It is bifidogenic, highly solubile and advantageous during heat processing, as it reverts to oligofructose rather than monosaccharides. Inulin shows high persistence along the human large intestine promoting the stimulation of bifidobacteria in the distal colon and improves mineral bioavailability of calcium and magnesium.

Inulin has been shown to contribute to a normal digestive function and may help to maintain healthy blood cholesterol level, maintain healthy postprandial glucose level and to maintain healthy bowel function.

### XOS

XOS is an emerging prebiotic with low degree of polymerisation, generally derived from corn cob, sugarcane bagasse, wheat straw, rice straw, rice husk derived.

XOS comprises β 1-4 xylose linkage molecules and has been shown to be bifidogenic, have a low DP and active in the proximal colon. It is high soluble and improves mineral bioavailability for calcium, magnesium, zinc and iron.

XOS has a number of advantages when compared to other prebiotics. It is efficient at aiding the proliferation of beneficial bacteria and only requires a low dose to increase Bifidobacterium. XOS is selective for the proliferation of: B. adolescentis, B. longum, L. salivarius. XOS is heat and acid stable, has a low moisture activity, a low viscosity and results in low gas production.

### Mg

Mg is an essential mineral suitable for supplement and food applications and serves as a cofactor for over 300 enzymes involved in biosynthetic processes. It is part of the Mg-ATP complex, is essential for oxidative phosphorylation and has roles in energy metabolism, mineral homeostasis, calcium metabolism, and neuromuscular and endocrine function.

In the human body, 50 to 60% of Mg is located in the bone. Part of it is readily exchangeable with serum and therefore bone represents a Mg store. The remaining Mg is mainly intracellular; extracellular magnesium represents only 1% of the total Mg content of the body.

Mg contributes to: a) a reduction of tiredness & fatigue; b) electrolyte balance; c) normal energy-yielding metabolism; d) normal functioning of the nervous system; e) normal muscle function; f) normal protein synthesis; g) normal psychological function; h) the maintenance of normal bones; i) the maintenance of normal teeth; and j) has a role in the process of cell division. Animal model studies have suggested that Mg itself can promote gut barrier integrity and upregulate the concentration of health positive gut microbes such as Lactobacillus (Xia, Y., et. al., (2022) Front Immunol. 13: 874878).

### Evaluation of Dietary Fibres

The impact on faecal microbiome composition and activity of Inulin and XOS, together with a selection of structurally diverse bifidogenic dietary fibres and prebiotics was evaluated in a series of in vitro faecal culture experiments.

Non pH-controlled, anaerobic, faecal cultures were investigated to evaluate the rate of gas production and cumulative gas production over 24 h in the presence of each test substrate.

pH-controlled, anaerobic, faecal cultures were carried out to evaluate the impact of each test carbohydrate on gut microbiome composition and activity (organic acid production). The test carbohydrates were as follows: Inulin, Fructooligosaccharides (FOS), Xylooligosaccharides (XOS), gentiooligosaccharides (GeOS) and cellobiose.

All carbohydrates were tested in parallel, using the same faecal sample from a healthy donor. Fermentations were repeated using the faecal samples of each of 6 donors. Faeces in the absence of test substrate were used as the negative control in each run.

With reference to FIG. 1, the experiments concluded that the non pH-controlled cultures showed that XOS fermentations produced the lowest rate and cumulative gas production. For inulin, the rate of gas production showed a more prolonged gas evolution pattern suggesting slower fermentation. The rate of gas production for cellobiose was significantly higher compared to all test substrates.

With reference to FIG. 2, the experiments concluded that for the pH-controlled cultures, significant increases were observed in Bifidobacterium and Lactobacillus with all test substrates compared to the negative control (faeces).

Inulin supported further growth in Bifidobacterium & Lactobacillus between 10 and 24 h suggesting prolonged fermentation activity. Inulin supported significant increases in Faecalibacterium prausnitzii numbers at 24 h, whilst no significant effect was observed in the pathogenic group of Clostridium histolyticum.

With reference to FIG. 3, the experiments concluded that for pH-controlled cultures, high levels of lactate were produced with XOS, Cellobiose, GeOS and FOS, and this reflects the high fermentation rates leading to lactate accumulation. Lactate accumulation was significantly lower in inulin cultures suggesting it is slowly fermented. Acetate was the main SCFA produced by all test substrates. Inulin produced significantly higher levels of propionate compared to all test substrates.

### Conclusions

In vitro faecal fermentation experiments suggest both XOS and Inulin have a significant impact in enhancing faecal Bifidobacterium numbers.

Lactate accumulation profiles suggest that XOS is rapidly utilised by lactate producing bacteria such as bifidobacteria, whereas inulin is utilised at significantly slower rates.

The results of the experiments suggest that inulin and XOS are good candidates to combine so as to support Bifidobacterium enrichment in the human colon, as XOS is rapidly fermented and can enhance Bifidobacterium levels in the proximal-transverse colon and the slowly fermented inulin can support their populations from transverse to the distal colon.

Feeding bifidobacteria throughout the colon can have a number of beneficial effects, such as reducing pathogenic clostridia in the proximal colon and increasing stool frequency.

It is known that there is a significant decline in Bifidobacterium concentrations and Mg levels in aging individuals. This can be due to a number of factors, such as physiological changes (e.g. loss of taste and smell), increased satiety, dietary choices and malnutrition, use of antibiotics and other prescription drugs and changes to physical activity and hormone levels. Therefore the composition of the present invention, could be utilised to maintain Bifidobacterium concentrations in aging individuals and increase the bioavailability of Mg. The addition of Mg could further enhance the health benefits for the individual and would be readily available for absorption.

It is also believed that Bifidobacterium concentrations decline in individuals who are undergoing athletic training (whether amateur or professional) and who are typically on a high protein diet. The composition of the present invention, could be utilised to maintain Bifidobacterium concentrations and increase bioavailability of Mg in individuals who are undergoing athletic training and who are on a high protein diet. The composition could be incorporated into a protein bar or milkshake so as to help counterbalance the reduction in Bifidobacterium concentrations due to the high protein diet and increase the bioavailability of Mg. The addition of Mg could further enhance the health benefits for the individual and would be readily available for absorption.

The composition of the present invention could be utilised to maintain Bifidobacterium concentrations and reduce proteolysis in those aging individuals. In particular, the composition of the present invention, could be utilised to promote saccharolytic fermentation throughout the colon and to reduce proteolysis. Proteolysis produces cellular toxins, carcinogenic and precarcinogenic compounds (ammonia, amines, indoles, phenols and hydrogen sulphide), which further damage the gut barrier. The composition of the present invention could be employed to reduce the formation of proteolysis metabolites and enhance their clearance from the body by supporting a regular bowel habit. The promotion of saccharolytic fermentation is applicable to athletes because of high protein intake and insufficient fibre in their diet. The promotion of saccharolytic fermentation is also applicable to aging individuals as studies report that in comparison to young adults, the faecal microbiota of non-institutionalised elderly showed an increase in proteolytic potential, but decreased saccharolytic potential with a low abundance of genes encoding steps in short-chain fatty acids production pathways.

Enhancing Bifidobacterium and saccharolytic fermentation promotes the SCFA formation, thereby lowering the pH of intestinal luminal contents. Greater acidity in the colon is thought to prevent minerals like calcium and magnesium from forming insoluble complexes in the gut. The release of magnesium from these molecules increases the mineral's bioavialability

The composition of the present invention could be utilised to maintain Bifidobacterium concentrations and reduce proteolysis in those aging individuals. In particular, the composition of the present invention, could be utilised to promote saccharolytic fermentation throughout the colon and to reduce proteolysis. Proteolysis produces cellular toxins, carcinogenic and precarcinogenic compounds (ammonia, amines, indoles, phenols and hydrogen sulphide), which further damage the gut barrier. The composition of the present invention could be employed to reduce the formation of proteolysis metabolites and enhance their clearance from the body by supporting a regular bowel habit. The promotion of saccharolytic fermentation is applicable to athletes because of high protein intake and insufficient fibre in their diet. The promotion of saccharolytic fermentation is also applicable to aging individuals as studies report that in comparison to young adults, the faecal microbiota of non-institutionalised elderly showed an increase in proteolytic potential, but decreased saccharolytic potential with a low abundance of genes encoding steps in short-chain fatty acids production pathways.

### Example 2—Combination Formulations of Inulin and XOS and Mg

The following formulations are examples of formulations which may be prepared and consumed as a formulation dose in a capsule, tablet or powder form or a pre-blend for subsequent formulation with a food product such as a protein bar or milkshake.

### Example Formulation A: Inulin and XOS and Mg

5 g inulin
1.4 g XOS
56 mg Mg

### Example Formulation B: Inulin and XOS and Mg

3 g inulin
1.5 g XOS
250 mg Mg

### Example Formulation C: Inulin and XOS and Mg

3 g inulin
1.4 g XOS
200 mg Mg

### Example Formulation D: Inulin and XOS and Mg

5 g inulin
1.5 g XOS
350 mg Mg

### Example Formulation E: Inulin and XOS and Mg

2 g-12 g inulin
1.4 g-8 g XOS
55 mg-250 mg Mg

### Example Formulation F: Inulin and XOS and Mg

2 g inulin
1.4 g XOS
56.25 mg

### Example Formulation G: Inulin and XOS and Mg

12 g inulin
8 g XOS
350 mg Mg

All of the above example formulations are intended to be administered once a day with water so as to provide a daily dose of each of the components. However, if desired, the formulations could be adapted so as to form multiple doses which collectively form the same daily dose of the components and the skilled addressee will appreciate that above formulations will simply be divided by the number of preferred daily doses.

Individual doses may be as follows:

### Example Formulation H: Inulin and XOS (High Inulin Dose) (8.3 g)

6.4 g HSI inulin=77%
1.4 g XOS=16.9%
0.5 g MgCl₂=6.0%

### Example Formulation I: Inulin and XOS (Medium Inulin Dose) (5.1 g)

3.2 g HSI inulin=62.7%
1.4 g XOS=27.5%
0.5 g MgCl₂=9.8%

### Example Formulation J: Inulin and XOS (Low Inulin Dose) (4.1 g)

2.2 g HSI inulin=53.7%
1.4 g XOS=34.1%
0.5 g MgCl₂=12.2%

In taste tests, Example Formulations H-J were dissolved in 250 ml of water at room temperature and pressure. Formulation I had the most neutral taste, whereas Formulation H had a slight sweet taste and Formulation J had a slight salty taste. In a study, 60% of participants preferred the medium inulin dose of Formulation I and reported: no bitterness; less lingering sweet aftertaste; less metallic aftertaste; and a balanced flavour profile.

The optional features set out herein may be used either individually or in combination with each other where appropriate and particularly in the combinations as set out in the accompanying claims. The optional features for each aspect or exemplary embodiment of the invention as set out herein are also to be read as applicable to any other aspect or exemplary embodiments of the invention, where appropriate. In other words, the skilled person reading this specification should consider the optional features for each exemplary embodiment of the invention as interchangeable and combinable between different exemplary embodiments.

Attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, and drawings), and/or all of the steps of any method or process so disclosed, may be combined in any combination, except combinations where at least some of such features and/or steps are mutually exclusive.

Each feature disclosed in this specification (including any accompanying claims, and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, and drawings), or to any novel one, or any novel combination, of the steps of any method or process so disclosed.

1. A composition comprising xylooligosaccharides (XOS), inulin and magnesium (Mg).

2-12. (canceled)

13. The composition of claim 1, wherein the inulin is derived from chicory.

14. The composition of claim 1, wherein the XOS is derived from maize.

15. The composition of claim 1, wherein the Mg is in the form of chloride, chloride 6 hydrate, citrate, dictrate or hydrogen citrate.

16. The composition of claim 1, wherein the inulin and XOS are present in a ratio in the range of about 4:1 to about 1:1.

17. The composition of claim 16, wherein the inulin and XOS are present in a ratio of about 2:1.

18. The composition of claim 1, wherein the inulin and XOS and Mg is present in a ratio of about 6.5:3:1.

19. The composition of claim 1, wherein the inulin is present in an amount in the range of about 0.6 g to about 4 g.

20. The composition of claim 1, wherein the XOS is present in an amount in the range of about 0.4 g to about 2.6 g.

21. The composition of claim 1, where the Mg is present in an amount in the range of about 18 mg to about 120 mg, or about 18 mg to about 85 mg, or about 0.25 g to about 0.75 g.

22-23. (canceled)

24. The composition of claim 1, wherein the composition is formulated so as to provide a daily dose of inulin in an amount in the range of about 2 g to about 12 g, or about 3 g to about 5 g.

25. (canceled)

26. The composition of claim 1, wherein the composition is formulated so as to provide a daily dose of XOS in the range of about 1.4 g to about 8 g, or about 1.4 g to about 1.5 g.

27. (canceled)

28. The composition of claim 1, wherein the composition is formulated so as to provide a daily dose of Mg in the range of about 55 mg to about 350 mg, or about 55 mg to about 250 mg, or about 0.75 g to about 2.25 g, or about 1 g to about 2 g.

29-32. (canceled)

33. The composition of claim 1, wherein the composition is in the form of a capsule and optionally wherein the composition further comprises an excipient or carrier compound to modify the release profile of the inulin and/or XOS through the intestinal environment.

34-45. (canceled)

46. A method of improving the gastrointestinal microbiota and maintaining and/or improving gut barrier integrity in an individual comprising administering a composition comprising xylooligosaccharides (XOS), inulin and magnesium (Mg) to the individual.

47. The method of claim 46, wherein the improved gastrointestinal microbiota increases the quantity of desirable microbiota.

48. The method of claim 47, wherein the microbiota comprises Bifidobacterium.

49. The method of claim 46, wherein the individual is a sports person or an individual undergoing athletic training or an individual undergoing a high protein diet regime.

50. The method of claim 46, wherein the individual is of an age of at least 45 years of age, or of least 60 years of age.

51. The method of claim 46, wherein the inulin is derived from chicory and/or the XOS is derived from maize.
